(19) United States
(12) Patent Application Publication
(10) Pub. No.: US 2009/0239901 A1
(43) Pub. Date: Sep. 24, 2009

(54) METHOD AND COMPOSITIONS FOR TREATMENT OF CEREBRAL MALARIA

(76) Inventor: Merouane Bencherif, Winston-Salem, NC (US)

Correspondence Address: WOMBLE CARLYLE SANDRIDGE & RICE, PLLC, ATTN: PATENT DOCKETING, P.O. BOX 7037, ATLANTA, GA, 30357-0037, US

(21) Appl. No.: 12/407,010

(22) Filed: Mar. 19, 2009

Related U.S. Application Data
(60) Provisional application No. 61/037,860, filed on Mar. 19, 2008.

(51) Int. Cl. A61K 31/444

(52) U.S. Cl. 514/305

(57) ABSTRACT

A method of treating inflammatory disorders, including cerebral malaria, by administration of modulators of α7 nAChR.

### BACKGROUND OF THE INVENTION

Evidence has recently emerged showing that the central nervous system (CNS) modulates the immune system through the reticuloendothelial system (RES). In the last five years, pioneering studies have shown that the α7 nicotinic acetylcholine receptor is at the apex of the “cholinergic anti-inflammatory pathway” which regulates key inflammatory cytokines responsible for the inflammatory debacle observed in conditions such as Alzheimer's disease and cerebral malaria (CM). This CNS modulation is mediated through the vagus nerve, utilizing the major vagal neurotransmitter acetylcholine (ACh) which acts upon α7 nAchR nicotinic receptors on macrophages.

The host responds to malaria infection with several strategies to target the parasite and protect its organs. These strategies are regulated by the balance between pro- and anti-inflammatory cytokines. However a deregulated response can also lead to the build-up of monocytes and lymphocytes in the small blood vessels of the brain. Together with red blood cells infected with malaria parasites, these monocytes and lymphocytes can compromise the integrity of the blood-brain barrier, thereby allowing cytokines like tumor necrosis factor alpha (TNF-α) and malarial antigens to enter the biochemical milieu of the brain and cause inflammation. Numerous studies provide evidence for the role of TNF-α in the pathogenesis of CM, and a relationship has been established between plasma concentrations of TNF-α levels and cerebral pathology. In experimental CM, TNF-β, now called lymphotoxin α (LT), was recently shown to be the principal mediator of cerebral pathogenesis. Indeed, LT and TNF-α belong to the same family, interact with a common receptor, and could act together during the pathogenesis of CM.

Anti-inflammatory actions of the α7 nAChR nicotinic receptor TNF-α and CM: Vagotomy increases LPS (lipopolysaccharide)-induced TNF-A serum levels and hepatic TNF-α responses. Electrical stimulation of the vagus nerve or treatment with ACh prevents the increased TNF-α release in vagotomized animals. The critical role of α7 nicotinic receptors in the modulation of TNF-α in LPS stimulated macrophages has been shown using antisense oligonucleotides to the α7 receptor. When the expression of α7 is blocked, ACh does not have an effect on LPS-induced TNF- a release. This observation has been extended to in vivo models which demonstrate that vagus nerve stimulation does not inhibit TNF- a release in α7 knockout mice. TNF-α is an early mediator of inflammatory responses and has been implicated in multiple inflammatory disorders such as diabetes, atherosclerosis, rheumatoid arthritis, sepsis and CM. Drug discovery efforts for these diseases have focused much energy on targeting TNF-α. As an understanding of the tissues involved in the cholinergic anti-inflammatory pathway from the CNS to the RES has unfolded, advances have also been made in understanding the molecular mechanisms involved.

It is desirable to develop a more complete understanding of the mechanistic rationale and the proof of concept for the use of novel drugs targeting the cholinergic anti-inflammatory pathway in the development of CM.

### SUMMARY OF THE INVENTION

Accordingly, one aspect of the invention relates to methods and compositions for treatment of inflammatory disorders including cerebral malaria by administration of modulators of α7 nAChR. The modulator is preferably an agonist. The agonist is preferably Compound A, (2S,3R)-N-(2-((3-pyridinyl)methyl)-1-azabicyclo[2.2.2]oct-3-yl)-5-methylthiophene-2-carboxamide or a pharmaceutically acceptable salt thereof, represented by Formula I below:

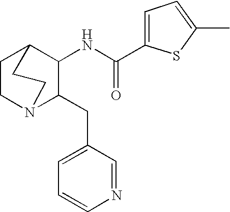

In other embodiments, the agonist is preferably Compound B, (2S,3R)-N-(2-((3-pyridinyl)methyl)-1-azabicyclo[2.2.2]oct-3-yl)benzofuran-2-carboxamide or a pharmaceutically acceptable salt of solvate thereof, represented by Formula II below:

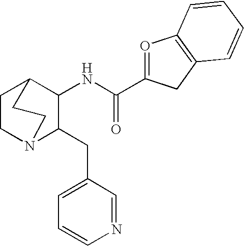

### BRIEF DESCRIPTION OF THE FIGURES

The following Figure descriptions and their corresponding Figures relate to particular embodiments of the present invention:

FIG. 1 show RT-PCR results demonstrating the presence of α7 and β2 subunits in cultured cells

FIG. 2A and 2B are a gel and graph, respectively, showing that preincubating cells with 10 mM α7 ligand can ameliorate radiation-induced up-regulation of IL-6 mRNA.

FIG. 3 shows gels and graphs reincubating cells with α7 ligand can ameliorate radiation-induced up-regulation of l-cam1 mRNA

FIG. 4 is a graph illustrating the effects nicotinic receptor α7 agonist Compound A on TNFα-induced production of VCAM-1 in human brain microvascular endothelial cells (EC).

FIG. 5 is a graph showing results of a study that indicates α7 agonists can prevent sepsis-induced death in rodents. Survival was monitored daily from day 0 to day 6. In the vehicle control group there was a 13% survival by day 6. In the animals treated with 0.003 mg/kg Compound B, there was a 13% survival by day (NS, p=0.360). In the animals treated with 0.03 mg/kg Compound B, there was also a 13% survival by day (NS, p=0.528). In the group treated with Compound B at 0.3 mg/kg, survival was greatly enhanced. Survival was 47% in this group, a significant improvement in survival (p=0.005).

FIG. 6 is a graph illustrating the effects 100 mM nicotinic receptor α7 agonist Compound A on TNFα-induced production of ICAM-1 in human brain microvascular EC.

FIG. 7A and 7B are graphs showing that Compound A promotes hippocampal neurogenesis in vivo and prevents microglial inflammation in vivo. Animals were treated with either vehicle and 8% 2H₂O label for 1 week. LPS (1 mg/kg, i.p) was injected on days 1, 3 and 5 of drug treatment and label. Microglia were isolated from brain and sorted by flow cytometry as CD11b+, F4/80+ cells. DNA was isolated from sorted microglia cells, hydrolyzed and derivatized for GC/MS analyses.

### DETAILED DESCRIPTION

Data gathered on α7 nAChR-mediated anti-inflammatory effects in the microvasculature in connection with the present invention indicates that the α7 nAChR is an attractive target for anti-inflammatory therapeutics in the treatment of CM. The studies underlying the present invention have demonstrated anti-inflammatory effects of α7 nAChR agonists in the brain and the vasculature, through the modulation of inflammatory cytokines, such as TNF, and of adhesion molecules, such as ICAM-1 and VCAM-1, in a number of in vitro and in vivo models including microglial inflammation, radiation-induced inflammation, and sepsis.

The inventor's studies have shown that a new chemical entity, the α7 nAChR selective agonist Compound A significantly inhibits TNF expression, decreases the levels of ICAM-1 and VCAM-1 expression in TNF-treated human brain microvascular endothelial cells (MVECs) and overcomes insulin resistance in whole animals. In addition, the inventors have found that the Janus kinase 2 (Jak2)-specific inhibitor AG-490 attenuates the effects of Compound A both in vitro and in vivo, thereby suggesting a role of Jak2 in α7-induced signaling events, potentially leading to protection against CM. All of these events were reported to be important in the development of Cerebral Malaria (CM). In addition, these events were attenuated by the Jak2—specific inhibitor AG-490 both in vitro and in vivo suggesting a role of Jak2 in a7 nAChR—induced signaling events leading to the protection against CM.

Without wishing to be bound by any particular theory, it is believed that the α7 nAChR—induced activation of Jak2 protects against cerebral malaria through 1) a decreased expression of pro-inflammatory cytokines 2) a decreased expression of the adhesion molecules ICAM-1 and VCAM-1 and 3) an increase in insulin sensitivity of the brain vasculature in malaria-infected mice.

The foregoing hypothesis can be verified by determining if the activities of the α7 agonist Compound A in brain MVECs in vitro are mediated through the cholinergic anti-inflammatory reflex induced via the α7 nAChR, involving the activation of Jak2, leading to the eNOS-mediated production of NO; and/or by determining if 1) the Jak2-PI3K-Akt pathway and/or 2) the Jak2-Src-eNOS pathway, both of which lead to the activation of eNOS, account(s) for the inhibitory effect of Compound A on TNF- or LT-α-induced ICAM-1 expression. Results from such studies can provide insights into the α7 nAChR regulation of pro-inflammatory cytokines and adhesion molecule expression in brain microvascular tissue.

Compound A is a new chemical entity selective for the α7 nAChR with a high binding affinity to membrane preparations from rat brain. An approximate thousand-fold separation exists between the affinities for the α7 and α4β2 receptor subtypes. In a Novascreen receptor binding profile on more than 60 receptors and enzymes, Compound A didn't interact with any other receptors with IC50<10 micromolar, providing a 100-1000 separation with other targets.

### Example 1

Quantifying the cerebral micro vascular expression of pro-inflammatory cytokines, levels of ICAM-1 and VCAM-1, insulin resistance and endothelial nitric oxide synthase (eNOS) expression (a marker of endothelial function) in α7+/+ and α7−/− mice infected with Plasmodium berghei ANKA (PbA).

If anti-CM effects of Compound A are mediated through the cholinergic anti-inflammatory reflex, Compound A should increase eNOS expression, inhibit pro-inflammatory cytokine and adhesion molecules expression in brain micro vascular tissue and inhibit the development of CM in the PbA infected α7+/+ mice. These effects, however, will be attenuated in the PbA infected α7−/− mice. The α7−/− mice have an exaggerated inflammatory response to endotoxin, but no studies have investigated whether they also have an increased susceptibility to CM. These mice exhibit normal growth, survival, gait and anatomy, and have no significant developmental or neurological abnormalities. Therefore, they represent an appropriate model to examine the role of the cholinergic anti-inflammatory pathway on PbA-induced inflammation and the development of CM.

### Example 2

Compound A protection from experimental CM in PbA infected mice occurs through the Jak2 activation.

A Tamoxifen-induced Jak2−/− mouse model infected with PbA can be used to quantify the cerebral micro vascular expression of pro-inflammatory cytokines, the levels of ICAM-1 and VCAM-1, insulin resistance plus eNOS expression An increase eNOS expression, inhibition of pro-inflammatory cytokine and adhesion molecules expression in brain micro vascular tissue and inhibition of the development of CM in the PbA infected Jak2+/+ mice; with attenuation of these effects in the PbA infected Jak2−/− mice by Compound A indicates that the anti-CM effects of Compound A are mediated through the cholinergic induced activation of Jak2.

### Example 3

The α7 nAChR and Inflammation

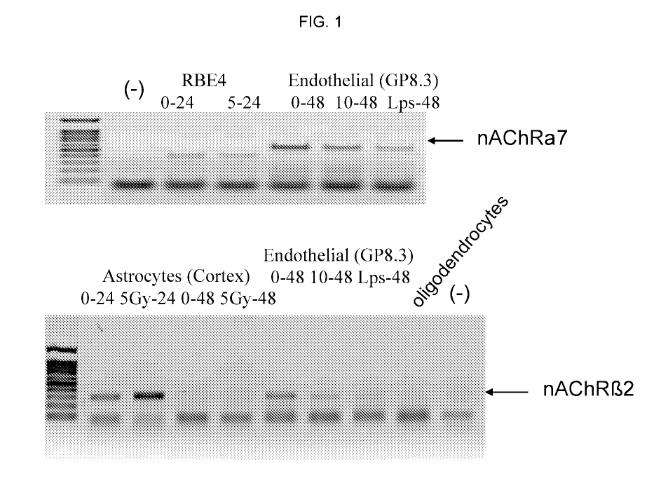

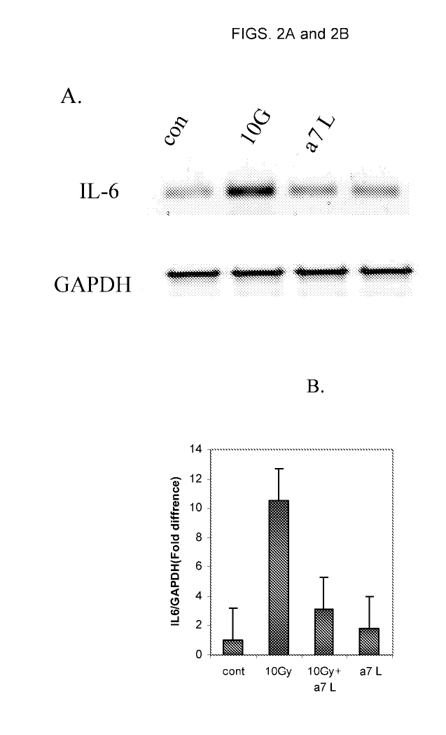

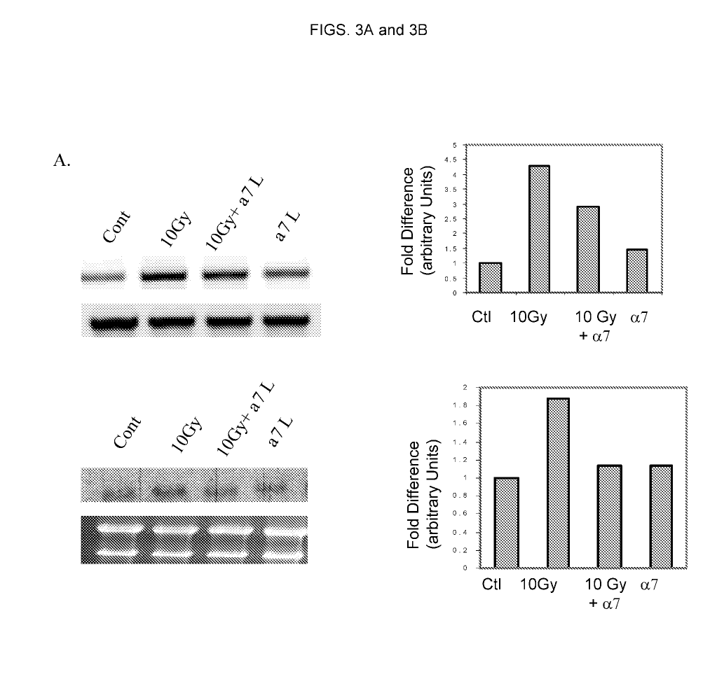

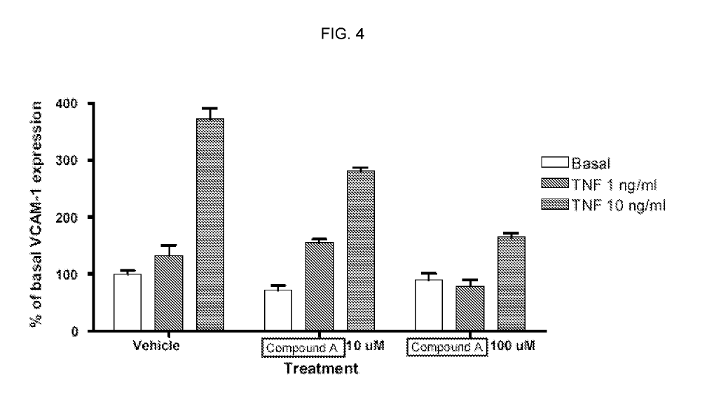

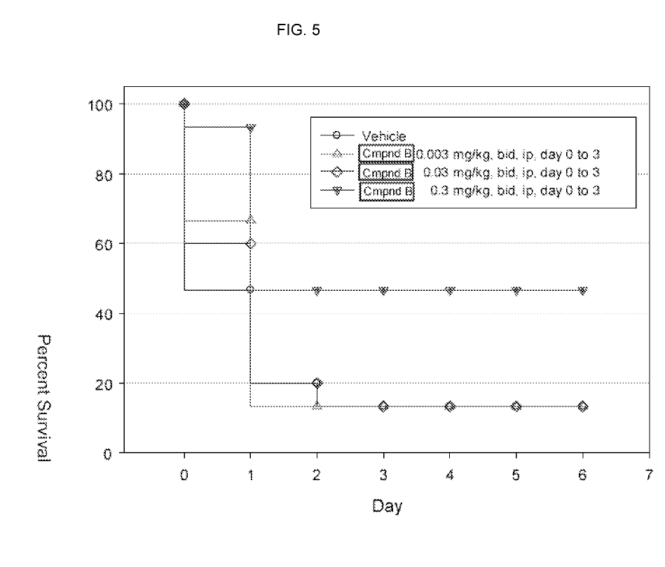

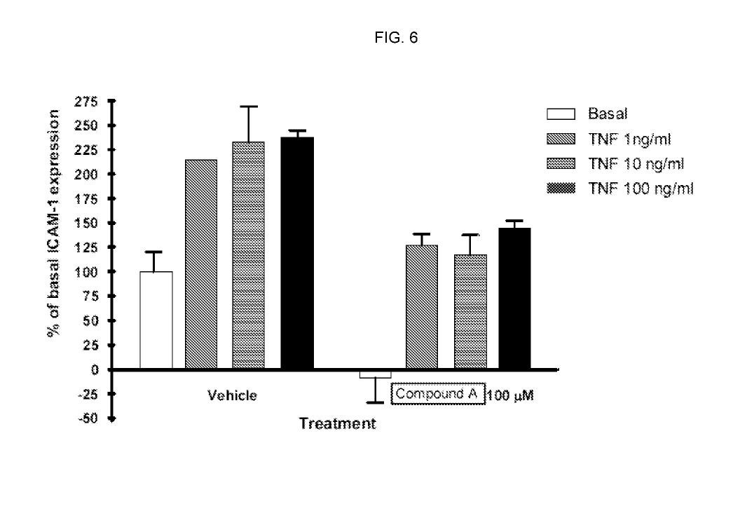

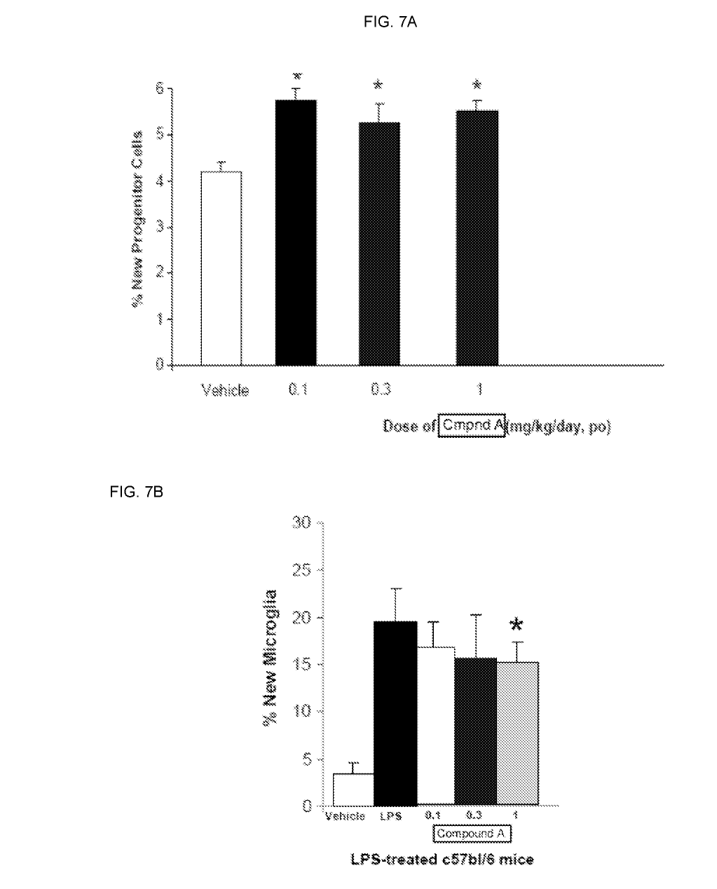

FIGS. 1-7 show that the α7 receptor is expressed in endothelial cells (FIG. 1); drugs targeting the α7 receptor prevent the proinflammatory cytokines induced by radiation (FIGS. 2 and 3); drugs targeting the α7 receptor inhibit TNF-A mediated V-Cam and I-Cam activation in endothelial cells (FIGS. 4 and 6); drugs targeting the α7 receptor inhibit mortality induced by sepsis in rodents (FIG. 5); and drugs targeting the α7 receptor prevent the microglia inflammation induced by LPS in vivo (FIG. 7) Together, these data indicate that Compound A can be used for the treatment of cerebral malaria.

What is claimed is:

1. A method of treating inflammatory disorders by administration of a modulator of α7 nAChR.

2. The method of claim 1, wherein the modulator is an agonist.

3. The method of claim 2, wherein the agonist is (2S,3R)-N-(2-((3-pyridinyl)methyl)-1-azabicyclo[2.2.2]oct-3-yl)-5-methylthiophene-2-carboxamide, represented by Formula I below, 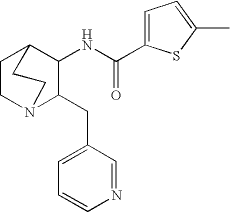
or a pharmaceutically acceptable salt or solvate thereof.

4. The method of claim 2, where the agonist is (2S,3R)-N-(2-((3-pyridinyl)methyl)-1-azabicyclo[2.2.2]oct-3-yl)benzofuran-2-carboxamide, represented by Formula II below, 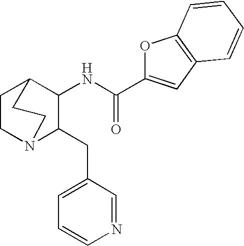
or a pharmaceutically acceptable salt of solvate thereof.

5. The method claim 1, wherein the inflammatory disorder is cerebral malaria.
